(19) United States
(12) Patent Application Publication
(10) Pub. No.: US 2007/0168224 A1
(43) Pub. Date: Jul. 19, 2007

(54) ADVANCED DIABETES MANAGEMENT SYSTEM (ADMS)

(76) Inventors: Alan M. Letzt, Purcellville, VA (US); Jacob Lefkowitz, Springfield, VA (US)

Correspondence Address: WOOD, HERRON & EVANS, LLP, 2700 CAREW TOWER, 441 VINE STREET, CINCINNATI, OH, 45202, US

(21) Appl. No.: 11/562,663

(22) Filed: Nov. 22, 2006

Related U.S. Application Data
(60) Provisional application No. 60/738,594, filed on Nov. 22, 2005.

(51) Int. Cl. G06Q 10/00; A61B 5/00

(52) U.S. Cl. 705/2; 600/300

(57) ABSTRACT

An automated healthcare communication system that facilitates communications between healthcare providers and patients and provides timely data to providers to help patients self manage their diabetes. Messages are presented to patients at pre-selected times to help them learn about the disease and develop appropriate behaviors that improve their health and to prompt them for data or for answers to specific questions or surveys. The system applies advanced speech recognition technology to make the system easy for patients to use and understand via a telephone network. Data collected from the patient is presented in real-time to healthcare providers so the patients' progress can be monitored and treatment plans adjusted accordingly.

### RELATED APPLICATIONS

This application claims the benefit of U.S. Provisional Application No. 60/738,594, entitled “ADVANCED DIABETES MANAGEMENT SYSTEM (ADMS), filed Nov. 22, 2005, which application is incorporated herein by reference in its entirety.

### FIELD OF THE INVENTION

The present invention is generally directed to a system and method for providing healthcare to patients and specifically for interfacing with patients via telephone, Internet and speech recognition technologies for managing particular ailments that need periodic oversight and patient education.

### BACKGROUND OF THE INVENTION

Various health ailments and conditions suffered by patients and handled by healthcare providers are categorized as chronic. Thus, they require ongoing treatment and attention over time. For example, certain chronic diseases require continuous treatment and oversight of the patient's condition for the overall health of the patient. Diabetes is one of the most common chronic diseases in the United States. Type 2 diabetes (also known as “non-insulin-dependent diabetes”) currently affects approximately 18.2 million Americans and that number increases each year. Factors that influence the increased incidence of Type 2 diabetes include poor diet and lack of exercise. As such, treatment regimens for the disease often focus on diet monitoring and discipline, as well as an exercise plan or program.

For example, patients with Type 2 diabetes usually are asked to follow a treatment plan that affects their diet and/or exercise. Such plans may be adjusted weekly or monthly, or in some other desired timeframe. Thus, the quantitative measures for Type 2 diabetes are most useful when trends are reviewed by healthcare providers over longer time periods.

One possible treatment plan for a patient with a chronic ailment would involve routine personal visits with a healthcare provider for the patient to be examined, to review their treatment program, to review their attention and commitment to the program, as well as to provide any data, such as blood glucose readings, which may be needed for treatment. For example, such a regimen might involve initial educational programs or classes put on by a healthcare provider, with the distribution of educational materials to help and empower patients to better manage their diabetes. At each of the classes, during different intervals, the patients may review how well they have achieved their goals and may discuss a log of their blood glucose levels, which would be brought to class. While such a treatment regimen is suitable for initially educating and advising those that suffer from Type 2 diabetes, the cost of continuous personal visits with healthcare providers can be quite high and, thus, cost prohibitive. Furthermore, there are often long intervals between such visits and classes.

As may be appreciated, time constraints, costs, convenience issues, and the availability of suitable healthcare educators, are various factors which limit the increased frequency of such educational classes or programs. However, since Type 2 diabetes and its management effectively become a lifestyle program, frequent educational reinforcement, goal and compliance tracking, and monitoring of blood glucose levels are desired.

Accordingly, it is desirable to improve upon treatment regimens and plans for addressing chronic diseases. One particular desirable goal is to improve upon such treatment plans for Type 2 diabetes, but other chronic ailments as well will benefit from an improved regimen that is convenient, cost-effective, and does not require a significant amount of time from a patient's schedule. It is further desirable to provide a treatment regimen that does not require the presence of a healthcare provider, is convenient for the patient, and which may be specifically tailored for the patient's lifestyle and schedule. These concepts and others are addressed by the present invention as discussed in further detail below.

### BRIEF DESCRIPTION OF THE DRAWINGS.

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a block diagram of one embodiment of a system for practicing the invention.

FIG. 2 is a illustrative diagram of a sample website setup page to be utilized in an embodiment of the invention.

FIG. 3 is an illustrative diagram of a sample patient record page to be utilized in an embodiment of the invention.

FIG. 4 is an illustrative diagram of another sample patient record page to be utilized in an embodiment of the invention.

FIG. 5 is an illustrative diagram of a sample scheduling page to be utilized in an embodiment of the invention.

FIG. 6 is a block diagram of one embodiment of a system for practicing the invention.

FIG. 7 is a flowchart diagram illustrating one embodiment of the invention.

FIGS. 8-10 illustrate exemplary sample reports in accordance with the aspects of the invention.

FIG. 11 is a block diagram of another embodiment of a system for practicing the invention.

FIG. 12 is a block diagram of another embodiment of a system for practicing the invention.

### DETAILED DESCRIPTION OF THE EMBODIMENTS OF THE INVENTION

The present invention includes a home health management system and method that applies Internet, telephony and speech recognition technologies in conjunction with data transfer and reporting methodologies to provide scheduled reminders, collect patient data, and to reinforce patient education. In one embodiment, the healthcare provider schedules messages, prompts, and/or educational information for the patient using a computerized interface and the Internet to configure a data center. A communication session is then established with the patient by the data center, such as with a telephone call to the patient. The patient receives voice messages, prompts and questions via the telephone or other communication device that are directed to the health condition of the patient, and the patient responds to the prompts/questions verbally using the telephone. The healthcare provider also monitors the patient's responses using the computerized interface and may generate reports based on such patient communication sessions and the data obtained therefrom.

In one embodiment of the invention, as discussed herein, this system allows healthcare providers to present a series of educational messages and reminders to patients diagnosed with Type 2 diabetes to help them to learn to manage their health condition and to reinforce their diabetes education and diet/exercise behaviors. The invention utilizes interactive messages that are presented to the patient in a verbal format over the telephone. In addition, the invention collects data from the patient in various ways, such as by asking questions over the telephone or prompting the patient to download data from a metering device, such as a blood glucose meter. Verbal responses from the patient to questions posed by the inventive system are interpreted by a speech recognition engine. The speech recognition engine then stores a computer-text data version of the response in a database.

Automated data collection is performed by the system to capture and store the patient's blood glucose readings. The patient uses a glucose meter that can be connected to a telephone modem, for example, to upload the blood sugar readings to a central database. These readings can then be viewed by the healthcare provider in a report format including graphs. Review of the trends in the reports by healthcare providers can lead to adjustments in the treatment plan to better help the patient manage the disease. Such adjustments might then be further managed through the present invention using further education of the patient.

Various embodiments of the present invention, as discussed further herein in some detail, are directed to a diabetes management system for managing chronic illnesses or conditions such as Type 2 diabetes. However, the present invention has particular usefulness with other chronic illnesses or conditions which may require an ongoing regimen or program and continuous monitoring of patient compliance and health conditions associated with such illness along with their respective treatment/management program. As such, the present invention is not specifically limited to only diabetes management. Therefore, utilization of the acronym ADMS to indicate an Advanced Diabetes Management System is not limiting with respect to the invention.

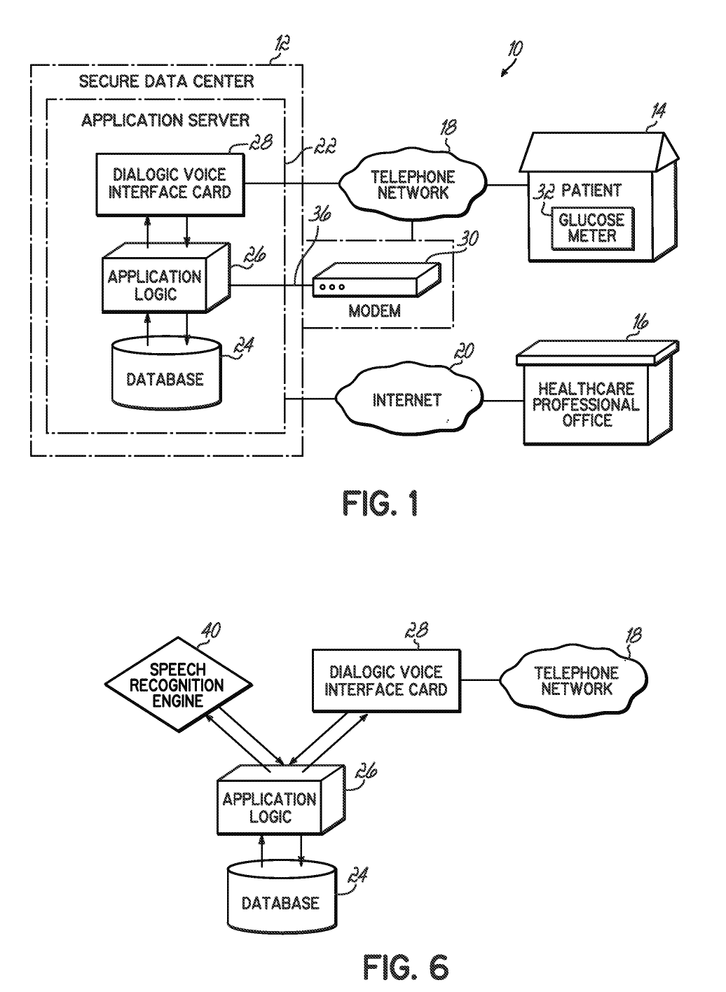

FIG. 1 illustrates one embodiment of a basic system for practicing aspects of the invention. In system 10, a data center 12 is configured to manage the process and interface with both a plurality of patients 14 through a communications network 18 and a plurality of healthcare providers 16, also through a communications network 20. Generally, the patients 14 may be located at home, which adds to the convenience of the invention, but the invention may also be utilized at locations away from home wherever a communications link is provided. Similarly, the healthcare providers 16, which might typically be healthcare professionals, such as doctors and nurses, may interface with the invention from their professional offices. However, other areas or locations that have a communications access may also be utilized, thus adding to the overall convenience and cost-effectiveness of the system 1 0. The term “healthcare providers,” as used herein, is not limited to traditional healthcare professionals such as doctors and nurses, but might also include dietitians or fitness experts to help and advise a patient in management of their diabetes or other disease.

In the illustrated embodiment of FIG. 1, a patient's interface to data center 12 is provided through a communications network 18 in the form of a telephone network. The healthcare providers access or interface with the data center through a communications network 20 in the form of an internet network, such as the public Internet. However, as discussed further hereinbelow, other forms of access to data center 12 might be utilized and other communications networks might be used.

Data center 12 utilizes one or more application servers 22, such as a standard computer server running the Microsoft WINDOWS Server operating system, for example. The data center 12 includes a database system 24 or database. The server 22 may run one or more database management systems 24, such as the Microsoft SQL Server application for accessing a database. Server 22 also runs the application logic software 26, that is configured and written to control the operation of the system 10 and data center 12, including delivery of scheduled voice messages and tutorials, collection of data, and report generation, as noted further hereinbelow.

To interface with the telephone communications network 18 for establishing a communication session with a patient, the data center 12, and particularly server 22, may use a voice interface card 29, (such as from Dialogic® Corporation) that provides a voice/audio connection and call-control capability from the server 22 to the telephone network 18. A telephone modem 30 provides a data communication interface between the server 22 and a glucose meter 32 for the patient, that may be located at the patient's home or some other location. For example, a Dex-2 glucose meter, available from Bayer, might be used to provide data from the patient to the data center 12. The telephone network 18 may be a public telephone network used to communicate with the patient 14, either through voice 34 over the telephone, or data 36 via the glucose meter 32 and modem 30.

The Internet communications network 20 is used by the healthcare provider 16 to access a suitable Internet site, such as a server sponsored website, to control the system through server 22 and to schedule communication sessions with patients. This portal can be used to schedule voice messages, tutorials and questions and data prompts to be delivered to the patient. The interface through network 20 might also be used to view reports of the data collected from the patient in response to such prompts in accordance with some aspects of the invention. The website might be hosted by server 22 or by another server (not shown) linked to server 22.

In accordance with several aspects of the present invention, the system 10 allows a healthcare provider or other person to schedule a telephone call to a patient to provide information to the patient, or to obtain data associated with the patient, such as to have the patient answer a selected series of questions or a survey, or to have the patient send data, such as from a metering device (e.g. glucose meter). Additionally, reminder messages can be left, such as to take a particular action, or to not forget an appointment, or to provide other data. Furthermore, the present invention may be utilized to inform a patient of a particular assessment by the healthcare provider, based upon the data that was received through a current or previous interface with the patient by system 10. The healthcare provider 16, through the Internet interface 20, may tailor the applications run by server 22 to affect the way in which the server schedules the communication session and interfaces with the patient, the way in which the applications 26 collect data, the way in which assessments of the data are reported, and the type of reports that are obtained by the healthcare provider from the data center 12.

To initiate telephone calls and communication sessions with patients, a healthcare provider 16, through the Internet 20, accesses data center 12, and particularly accesses server 22 and the application logic software 26 therein, to schedule the call. Information regarding one or more patients is stored in the database management system 24 by the healthcare provider. That information, such as the name of the patient and a telephone number, may be accessed from the database system 24. Through a website page available on the Internet 20 through server 22, a particular communication session with a patient 14 or a plurality of sessions may be selected and tailored by the healthcare provider 16 to be executed by data center 12. For example, data fields may be selected and instructions entered through the website to set up communication parameters for communication sessions with a plurality of patients to then be processed by the application logic software 26.

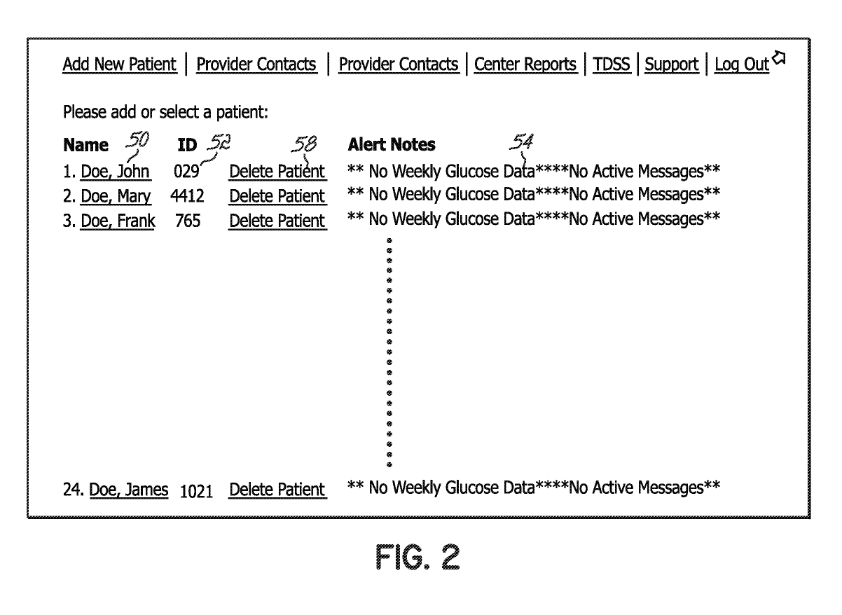

For example, FIG. 2 illustrates a website setup page run on server 22 that may be accessed by a healthcare provider for selecting patients, entering patient data, and setting up communication parameters for the patients that are used in various communication sessions with the patient. Illustrated in FIG. 2, names from a name field list 50 may be selected (e.g. by cursor) as part of a menu to schedule communication sessions. ID number 52 and alert fields 54 are provided to indicate additional information and communication status for a patient. Other control fields 58 might also be provided in the set-up page of FIG. 2.

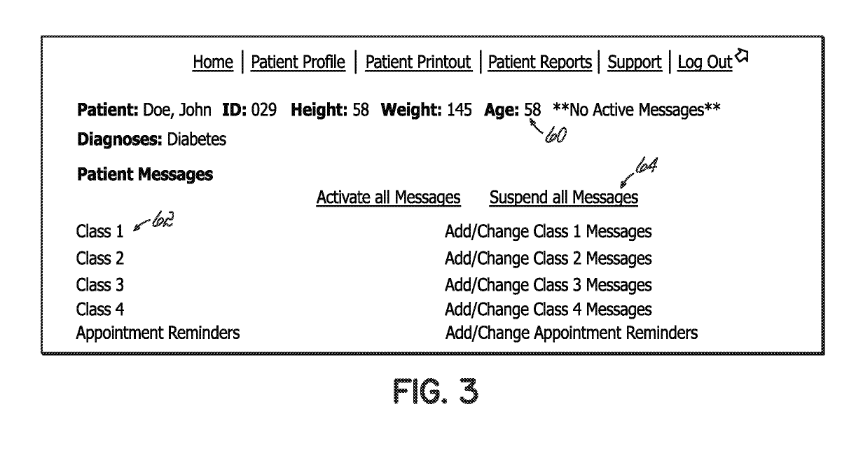

Once a patient is selected, a record for the patient is displayed as shown in FIG. 3. The record includes additional information 60 about the patient, such as their age/weight, diagnosis, and any allergies. Of course, other patient information might be displayed as well, other than that shown. A menu of data fields 62 is also selectable for entering parameters for a scheduled communication session based on a particular regimen or program (Class 1, Class 2, etc.) or to schedule appointment reminders. Other additions or revisions may be made for a particular patient's communication parameters using selectable fields 64. The communications parameters, as entered by a healthcare provider, are used for various purposes in a communication session. Particular selections might be activated for a patient. For example, communication sessions for Class 1 might be selected.

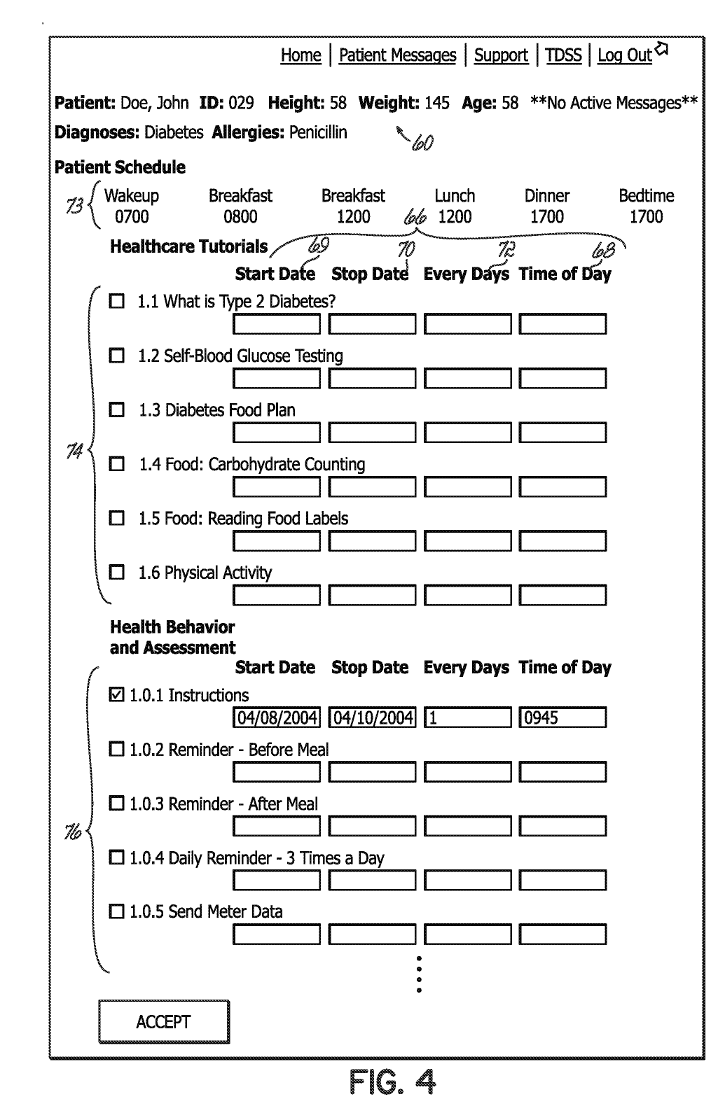

FIG. 4 shows a record associated with a particular selectable menu field or area of communication (e.g., Class 1) that may be used to set up and tailor communication sessions (e.g., phone calls to a patient) using data fields 66. A time might be selected for a particular scheduled telephone call through a Time of Day field 68. Also, other call parameters, such as the Start Date 69, Stop Date 70, and frequency (Every Day) 72 information might be entered through appropriate selectable fields. For scheduling purposes, the patient information of the record of FIG. 4 might also include particulars about a patient's schedule 73. Such scheduling information and parameters for a telephone call or other communication session, as provided and selected by a healthcare provider, is stored on server 22 and the database system 24. The scheduling parameters might be used to schedule educational messages that provide information to the patient and otherwise inform the patient. For example, using menu fields 74, educational messages or tutorials might be selected that reinforce class material from an earlier class, that reinforce health behavior, and that provide assessment messages that are related to the patient's goals with respect to their program or regimen. With menu fields 76, various reminders associated with a healthcare regimen might be scheduled.

In one embodiment of the invention, as noted above, the communication session is a telephone call to the patient. For providing tutorials and other educational information, a pre-recorded message might be played for the patient. Similarly, for reminders or questions, pre-recorded messages might be played. Alternatively, in those embodiments that use speech recognition to capture data from the patient, text-to-speech (TTS) features might be used to generate verbal messages, questions, and reminders.

Furthermore, in addition to educational or tutorial messages or reminders, a series of questions might be selected to be asked during the telephone call to the patient to prompt a patient for data associated with their health condition (e.g., diabetes). For example, the healthcare provider might be given the opportunity to select from a menu of various questions to ask the patient. Alternatively, a pre-arranged survey might be selected which includes a list of questions related to certain features of the disease or the treatment/management regimen. For example, the educational messages and tutorials, as well as the questions or surveys to be provided to the patient, are based upon the particular patient regimen that is entered on the Internet website by a healthcare provider, such as a nurse. The patient's regimen may be linked to the patient's goals, for example, in diet or exercise, for managing the diabetes.

One particular survey that might be utilized to prompt the entry of patient data is the Diabetes Self-Management Assessment Report Tool (D-SMART), which may be used to assess diabetes-related patient behavior. The D-SMART survey was developed for the American Association of Diabetes Educators (AADE) and is often used for patients who attend diabetes classes. Utilizing the present invention, the D-SMART version is provided in a telephonic form. The patients then provide the prompted data and respond to questions associated with the D-SMART survey and the questions are answered on the telephone during the scheduled phone call, in accordance with one aspect of the invention.

Other questions for prompting the patient to provide data might address issues such as: whether they exercised, how long, how frequently, what they ate, when they ate, did they stay on an eating schedule, alcohol consumption, medication use and adherence to schedule, blood sugar monitoring, blood tests, physical monitoring (e.g., foot tests), smoking, visits to a physician, stress management, motivation, etc. It will be understood by a person in the art that this list of topics/issues is not exhaustive as it relates to the invention.

In addition to educational information/tutorials and questions/surveys, system 10 also provides reminders to the patient to take certain actions, such as in their dietary or exercise routines. Furthermore, as discussed in greater detail hereinbelow with respect to the automated glucose meter 32 of the system, the patient may be prompted and reminded to provide blood glucose data to the data center 12.

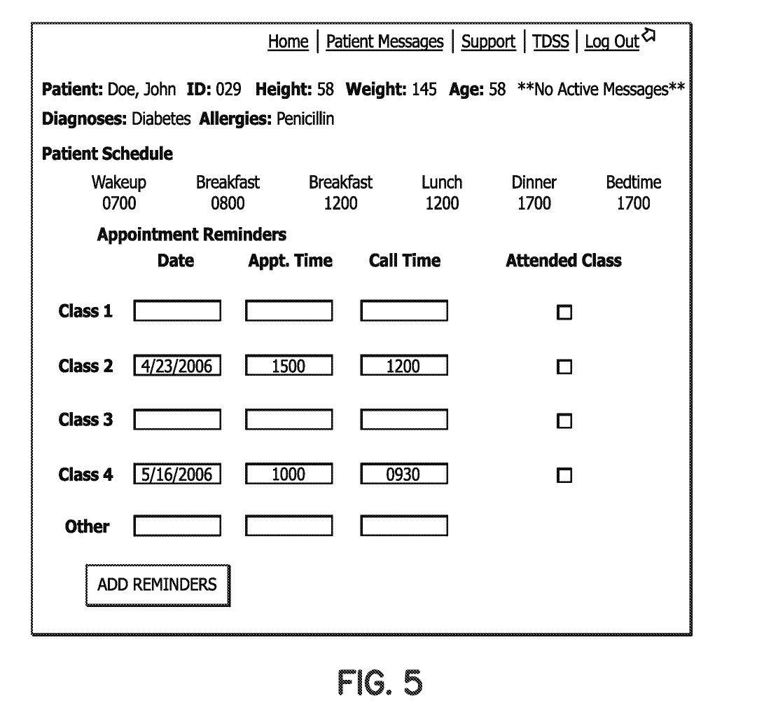

In another aspect of the invention, appointment reminders are provided to a patient in the communication sessions. Referring to FIG. 5, various appointments, such as to attend a class, may be scheduled. The system 10, then establishes communication with the patient appropriately, such as through a telephone call, to remind the patient. The healthcare provider, through Internet communication network 20 can tailor the parameters for the reminder communication sessions using selectable data entry fields as shown in FIG. 5. In these ways a communication session with a patient is scheduled and established according to selected parameters and the patient is provided educational information, reminders, or is prompted for information and data that is associated with the health condition or treatment of the patient. The invention empowers the patient to manage their condition without the presence or direct, in-person oversight of the healthcare provider.

Data center 12, and particularly server 22, is secure due to the nature of the present invention, and the storage of confidential patient information. Similarly, the website provided through Internet 20 and server 22 is also secure. For example, the inventive system 10 may be set up to be HIPAA compliant. The website server would be hosted at a secure location. Furthermore, the website for the system provided through Internet 20 would require a user name and password for access by a healthcare provider in accordance with HIPAA requirements. In another exemplary embodiment, the HTTPS secure transfer protocol might be used.

Once a patient communication session is set up on the secure website through selectable parameter fields and stored in the database system 24, the entered data is then accessed and utilized at the appropriate selected times, to initiate a communication interface and session through telephone network 18 and/or the Internet 20. The communication session is based on the rules of the application logic software 26 and the parameter information stored in the database system 24 for the patient. For example, a telephone call would be initiated to the patient through the voice interface card 28 and telephone network 18 at selected times. Then, in the communication session, the patient would be prompted for data associated with their health condition, such as to provide answers to questions or glucose meter data, or might be provided tutorials or reminders, for example.

For further security in the system, the patients may be required to initially call the data center 12 from a phone and activate a private pass code before calls can be placed to them by the system in accordance with the principles of the invention. That is, not only will a healthcare provider have to schedule the communication session, but the patient will have to agree to the interface through a call to data center 12, for example.

In accordance with another aspect of the invention, the application logic software 26 of server 22 records the results of the call. For example, software 26 knows if the call was answered, or not answered, or if the phone was busy. This information may be captured and then provided in a subsequent report to the healthcare provider. For example, if the patient is not responding to the phone calls, this might be noted in a report or otherwise available as date to notify the healthcare provider of a reluctant patient, who may not be complying with the desired regimen.

If the call is answered by the patient, the patient may again be required to enter a pass code for the purposes of security. Alternatively, other secure methods of identification might be utilized. If the patient security measures are met, the call proceeds. Any voice messages, such as tutorials and informational messages are then played to the patient. Similarly, any reminders might also be played for listening by the patient. Also, the patient is prompted for data in the form of answers to questions or the sending of metered data.

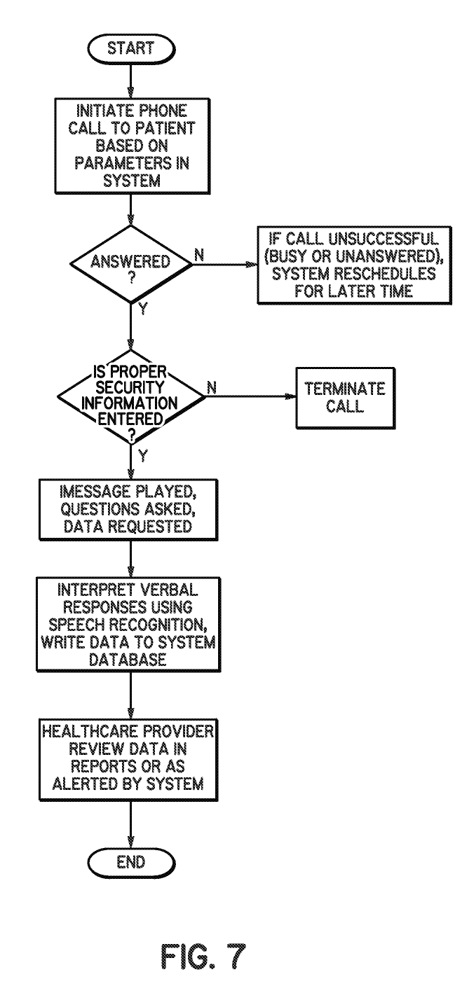

In accordance with one aspect of the present invention, if one or more questions are asked, or a survey is provided for the patient, system 10 captures the patient's responses either by telephone data entry, or by speech recognition. In one particular desirable embodiment of the invention, the patient's verbal responses to questions that are asked during the call are interpreted by a speech recognition feature of the invention. Referring to FIG. 6, a speech recognition engine 40 might be incorporated as part of the application logic software 26, or might be a separate application accessed by application logic software 26. The patient's verbal responses are interpreted by the speech recognition engine 40 and converted into text. For example, the speech recognition engine might have the ability to capture simple YES and NO questions, as well as to handle a certain size vocabulary from the speaking patient. The text responses are then recorded and written by application logic software 26 into the database system 24 for access and recall by a healthcare provider, such as to be used within one or more reports, as discussed further hereinbelow.

If the call is unsuccessful, such as it is not answered or a busy signal is perceived, the application logic software 26 reschedules the call for a later time based on a set of default rules, or a set of rules that might be set by the healthcare provider through selectable parameters.

In that way, patient communication sessions establish communication to further educate the patient, to instruct and remind the patient, and to obtain data from the patient in the form of metered data, as well as answers to specific questions. Tutorials and educational messages might be selected and grouped by type. For example, messages associated with different areas, such as health tutorials, patient goals, health behavior and assessment, and reminders might be played. In addition to reminders to take certain actions regarding exercise or diet, or to transmit data, such as blood glucose meter readings, reminder messages might also be played for reminding of appointments for a class or a meeting with a particular healthcare provider. As noted, in accordance with one aspect of the present invention, the content and frequency of the telephone calls and the messages can be individualized for each patient. Willing patients might be contacted less frequently than those which are more reluctant or forgetful.

While the messages and questions played to the patient might be pre-recorded, with the utilization of a speech recognition engine 40, text-to-speech (TTS) features of the speech recognition engine might also be utilized to generate educational messages and/or questions from text files. Generally, text-to-speech features would be most appropriate for questions, rather than longer thirty second or one-minute educational tutorials. In accordance with another aspect of the invention, questions might be intermingled within an educational/tutorial message such that the path through an educational message is dictated by the answer to a specific question using speech recognition.

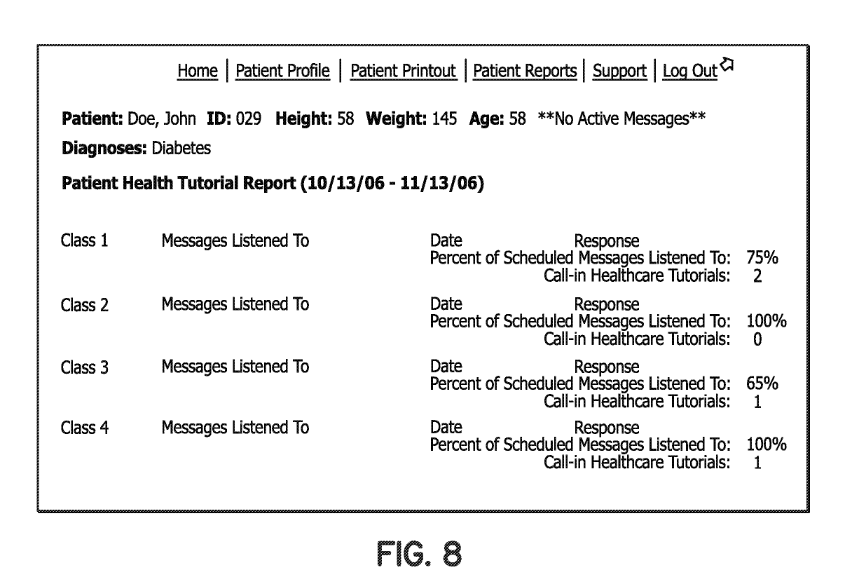

FIG. 7 provides an example flowchart of one exemplary scenario according to the aspects of the invention.

In addition to providing information to patients, data is also collected which may be utilized by the healthcare provider and may be implemented within one or more reports. Additionally, the system 10 of the invention may be utilized to provide further follow-up or alert interfaces with a patient based upon the acquired data. Through the Internet interface 20, the healthcare provider 16 may access information in the database system 24 of data center 12 in order to review the collected data, as well as to generate reports that may be reviewed and studied. Furthermore, the application logic software provides alerts to the healthcare provider on certain data of the patient that may indicate a serious condition that needs immediate attention. For example, as discussed further hereinbelow, blood glucose meter readings are provided through telephone network 18 and modem 30 to the server 22. As part of a patient regimen selected by the healthcare provider, a range of meter readings might be selected for an alert message. For example, high or low readings, or a target range may be selected for the blood glucose level. Once the data is collected, it may be selected for use in a report by the healthcare provider. For example, a report might be generated which shows and summarizes the number of high and low glucose levels that exceeded selected high and low set points. Such summary may be time specific, such as set for the last fourteen or thirty days, or whatever desirable parameters might be selected by the healthcare provider. In another report, the number of times that the blood glucose level was within the target range might also be reported, such as over a selected time period of fourteen or thirty days. In accordance with one aspect of the present invention, the data collected over the system 10 may be used in reports in various selectable forms.

In accordance with another aspect of the present invention, email messages, pager messages, or telephone calls might be utilized by the system 10 through the communication networks 18, 20 to contact the healthcare provider when the patient's data indicates a serious condition that needs immediate attention. For example, when blood glucose levels are out of an acceptable range or exceed high or low values, the healthcare provider might be notified by the system 10. Alternatively, if a patient is not responding to the telephone calls after numerous attempts, that information might also be provided to a healthcare provider by an alert telephone call, email message or page.

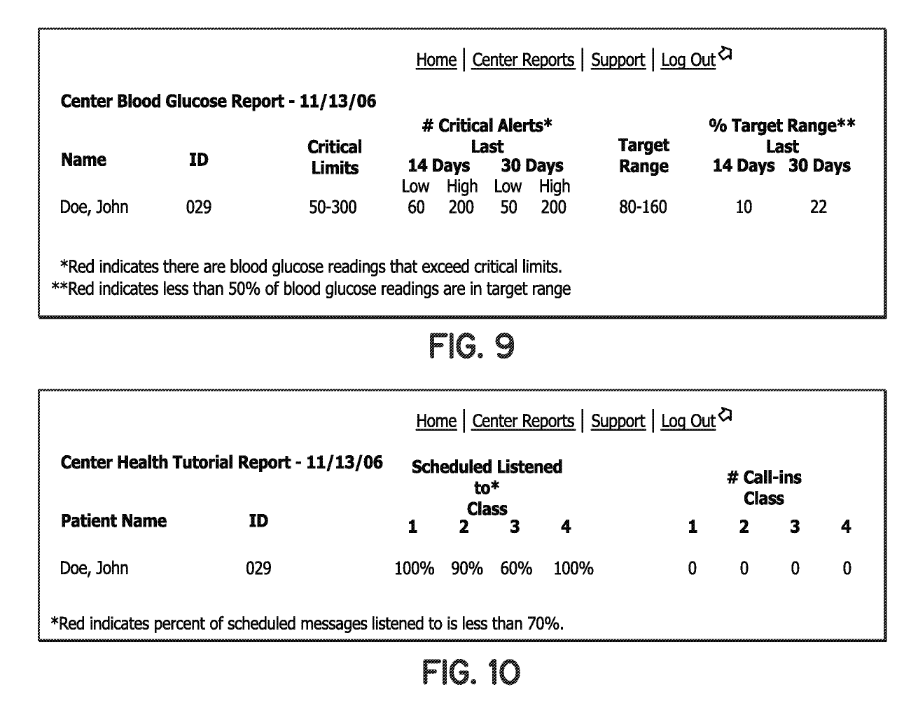

FIGS. 8-10 show exemplary sample reports with data collected from the patient communication sessions in accordance with the aspects of the system 10. FIG. 8 shows a Patient Health Tutorial Report with the percentages of messages that were listened to by the patient with respect to selected scheduled communication sessions, or call-in health care tutorials. Therefore, the patients provide reportable data to the system just by accepting phone calls and listing to a message. FIG. 9 lists data associated with blood glucose levels and target ranges and limits as well as High/Low and IN-RANGE occurrences. FIG. 10 lists data associated with an additional Health Tutorial Report. Other report formats, such as behavioral reports, patient goal reports, and scheduled appointment reports, might also be selected. As may be appreciated, the data collected using the present system may be configured in various reporting formats as selected by a healthcare provider via an interface with the server 22 through Internet 20 and a computer at the location of the healthcare provider 16. The reports may be run from specific date ranges.

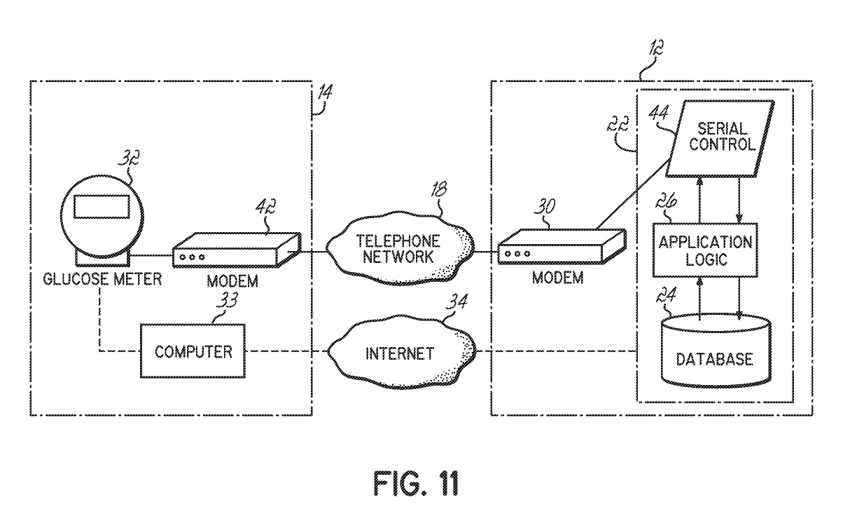

Turning now to the glucose meter data acquisition, FIG. 11 illustrates hardware components of the glucose meter uploading process according to one embodiment of the invention. In response to a voice message scheduled by the healthcare provider, the patient is instructed to upload glucose meter readings. As noted above, a suitable glucose meter 32 may be utilized at the location of the patient, such as the patient's home. The glucose meter 32 is coupled to a suitable modem 42. Once a message is heard and acknowledged, the patient connects the glucose meter 32 to the modem 42 that is specifically configured for interfacing with data center 12 through the telephone network 18. The glucose meter 32 is turned ON and automatically dials the telephone number for the modem 30 coupled to the server 22 at data center 12. Server 22 incorporates a suitable serial port control 44, which may be part of the operating system of server 22. The serial port control 44 interfaces with modem 30 and sends data to the application logic software 26. The application logic software controls receipt of the blood glucose readings and the associated transmission protocols for receiving the data. The application logic software stores the data from the meter readings in the database system 24. The data is then available for review on the website of system 10 by a healthcare provider, such as through the Internet 20. Also, the data is available for utilization in one or more reports, as illustrated in FIG. 9. The glucose meter data, for example, might be utilized to generate one or more alerts to the healthcare professional as noted above.

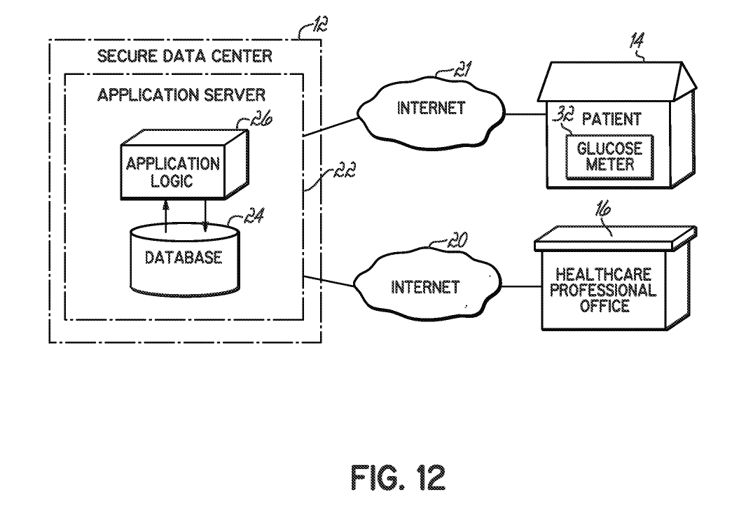

Other features are provided in alternative embodiments of the present invention. For example, while one embodiment utilizes input from the healthcare professional for scheduling and controlling calls to the patient, an alternative embodiment of the invention provides an interface for the patient, such as through an appropriate secure Internet website (see FIG. 12), that provides the patient with the ability to control the calls. For example, the patient might be able to stop the calls, or delay the calls, or otherwise tailor the patient regimen that was originally set up by the healthcare professional. The ability to stop or delay calls is particularly desirable when the patient is unavailable at the time initially scheduled by the healthcare professional. As such, the alternative embodiment of the invention allows the patient to self-schedule the telephone calls and the appropriate messages.

In another alternative embodiment of the invention, the patient is able to simply call in to system 10 through telephone network 18. Once security measures verify the patient's identity, they would be able to listen to any selected messages at their own convenience. Furthermore, the patients could select a menu of questions that would be answered, or a menu of tutorials that could be listened to.

In accordance with another aspect of the invention, the application logic software 26 monitors which messages the patient has heard and has not heard. If a patient has not heard a particular message, it may be rescheduled such as with a follow-up telephone call.

While information that is used to schedule and otherwise set up a patient regimen for use by the system may be obtained from paper records, in one desirable embodiment of the invention, the system is implemented with a server 22 that has access to electronic medical records so that information may be utilized in scheduling the patient regimens according to the aspects of the invention.

While one exemplary embodiment of the invention is discussed herein and illustrated in various drawing figures, the present invention might also be implemented using other technologies and other technology components within the system. For example, a communication session may be achieved with appropriate interface hardware and software using the Internet as the communication network for contact with the patient rather than a telephone network. As shown in FIG. 12, an Internet network 21 is used between the data center 12 and the patient 14. As illustrated in FIG. 12, rather than utilizing a telephone network for reminder calls, and educational tutorials that are broadcast in the telephone call, an email might be provided to a patient in response to a communication session set up by a healthcare provider where an email provides a link to a website page where the patient can read information, answer questions, and otherwise achieve what had been achieved in a telephone call in the previously-discussed embodiment of the invention.

Furthermore, a system as shown in FIG. 12 might still be utilized for telephone calls and voice connectivity between a patient 14 and the data center 12 utilizing Voice-over-Internet Protocol (VoIP) with appropriate hardware interfaces and software components, as understood by a person of ordinary skill in the art. Although FIG. 12 shows two different internet networks for the different patient and healthcare provider links, they may be the same internet network, such as the public Internet.

In still another embodiment of the invention, while exemplary embodiments discuss telephone calls to a patient's phone, that phone might be a traditional landline phone, or may include a cellular phone or other communications devices.

Furthermore, as illustrated in FIG. 12, if the Internet is utilized as an interface between the patient 14 and the data center 12, other communication devices, such as personal computers, personal data assistants (PDAs), and other types of suitable communication devices, might be utilized for the informational exchange in accordance with the principles of the invention. Also, utilizing the Internet as the data transmission vehicle between the patient 14, and the data center 12, may eliminate the use of modems, such as with the glucose meter readings. Rather, digital connectivity provides the data download from the glucose meter to server 22. The connectivity sessions would then occur in real-time and, in some embodiments, the data exchange may be protected using security measures, such as encryption. Furthermore, in certain embodiments of the invention, a process control may also employ software components, some portion of which may be embedded in a carrier wave and/or on a computer medium (such as memory, databases, disk or other storage medium). Signaling protocols, such as session-initiated protocol (SIP), and the like, may be used to establish, control, and facilitate various features of the invention.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details of representative apparatus and method, and illustrative examples shown and described. Accordingly, departures may be made from such details without departure from the spirit or scope of applicant's general inventive concept.

What is claimed is:

1. A method of providing healthcare to a patient, comprising the steps of:
establishing communication with a patient through a scheduled communication session set up by a healthcare provider;
prompting the patient during the communication session for data associated with a health condition of the patient;
recording at least one response to the prompting of the patient, including the data associated with the health condition of the patient; and
recalling data from the at least one response for review by a healthcare provider.

2. The method of claim 1 wherein the communication session includes a telephone call.

3. The method of claim 1 wherein the communication session includes an Internet session.

4. The method of claim 1 further comprising providing a menu, accessible by a healthcare provider over the Internet, the menu containing a plurality of selectable parameters for scheduling the communication session.

5. The method of claim 4 wherein the selectable parameters include at least one of the following: start date, stop date, communications frequency, and time of day for scheduling the communication session.

6. The method of claim 1 wherein the step of prompting includes transmitting a series of questions to the patient to be answered.

7. The method of claim 1 wherein answers to the questions are recorded using speech recognition to generate data associated with the health condition of the patient.

8. The method of claim 1 wherein the step of prompting includes reminding the patient to transmit data from a metering device used by the patient.

9. The method of claim 6 wherein the questions are related to one of diet, foot care, medication administration, or exercise actions of the patient.

10. The method of claim 1 wherein the health condition is diabetes and the patient is prompted for data associated with a diabetes condition of the patient.

11. The method of claim 8 wherein the health condition is diabetes and the step of prompting includes reminding the patient to transmit data from a glucose metering device used by the patient.

12. The method of claim 1 wherein recalling data includes generating a report based upon the data for review by a healthcare provider, caregiver, patient.

13. The method of claim 12 further comprising providing the report online over the Internet for review by the healthcare provider, caregiver, or patient.

14. The method of claim 1 further comprising providing an alert to a patient, caregiver or healthcare provider based upon the recorded data.

15. The method of claim 1 further comprising providing a menu, accessible by a healthcare provider over the Internet, the menu containing a plurality of selectable sets of questions for prompting the patient.

16. A method of providing healthcare to a patient, comprising the steps of:
through an Internet network, providing a menu to a healthcare provider, the menu containing a plurality selectable parameters for scheduling a communication session with a patient;
utilizing parameters selected by the healthcare provider, contacting a patient and conducting a communication session with the patient;
in the communication session providing, to the patient, at least one of educational information, reminders to take a particular action; or a prompt for a response from the patient.

17. The method of claim 16 wherein the educational information includes a tutorial that is played for the patient.

18. The method of claim 16 wherein the reminder to take a particular action includes a reminder about a future appointment with a healthcare provider.

19. The method of claim 16 wherein the reminder to take a particular action includes a reminder about at least one of a diet, foot care, medication administration, or an exercise regimen.

20. The method of claim 16 wherein the reminder to take a particular action includes a reminder about transmitting data from a metering device used by the patient.

21. The method of claim 16 wherein the prompt for a response includes transmitting a series of questions to the patient to be answered.

22. The method of claim 21 further comprising using speech recognition in the communication session to capture responses to the series of questions and record data associated with the responses.

23. The method of claim 16 wherein the communication session is conducted over a telephone.

24. The method of claim 16 wherein the communication session is conducted through an Internet network.

25. The method of claim 24 wherein the communication session includes one of an email or instant message.

26. The method of claim 16 wherein contacting the patient includes providing a link to a website.

27. A system for providing healthcare to a patient comprising:
a data center containing patient information and communication parameters for the patient;
a communication network operably coupled with the data center and with a location of a patient for establishing a scheduled communication session with a patient using the communication parameters of the data center;
the data center operable for prompting the patient, during the communication session, for data associated with a health condition of the patient and for recording at least one response to the prompting of the patient, including the data associated with the health condition of the patient; and
a communication network operably coupled with the data center and a location of a health care provider for providing data from the at least one response for review by a healthcare provider.

28. The system of claim 27 wherein the communication network operably coupled with the data center and with a location of a patient includes a telephone network and the communication session is over a telephone.

29. The system of claim 27 wherein the communication network operably coupled with the data center and a location of a health care provider includes an Internet network.

30. The system of claim 27 wherein the data center is further operable for providing a menu, accessible by a healthcare provider over the communication, the menu containing a plurality of selectable parameters for scheduling the communication session.

31. The system of claim 30 wherein the selectable parameters include at least one of the following: start date, stop date, communications frequency and time of day for scheduling the communication session.

32. The system of claim 27 wherein the data center prompts the patient for data by transmitting a series of questions to the patient to be answered.

33. The system of claim 32 further comprising a speech recognition engine for capturing and recording answers to the questions to generate the data associated with the health condition of the patient.

34. The system of claim 27 wherein the step of prompting includes reminding the patient to transmit data from a metering device used by the patient.

35. The system of claim 34 wherein the health condition is diabetes and the step of prompting includes reminding the patient to transmit data from a glucose metering device used by the patient.

36. The system of claim 27 wherein the data center is further operable for generating reports based upon the data for review by a healthcare provider.

37. The system of claim 36 wherein the communication network operably coupled with the data center and a location of a health care provider includes the Internet and the data center is operable for providing the report online over the Internet for review by the healthcare provider.

38. The system of claim 27 wherein the data center is further operable for providing an alert to a patient based upon the recorded data.

39. The system of claim 27 wherein the communication network operably coupled with the data center and with a location of a patient and a location of a health care is an Internet network.

40. The system of claim 39 wherein the communication session with a patient is over a telephone using Voice-over-Internet Protocol.

41. A system of providing healthcare to a patient, comprising:
a data center containing patient information and communication parameters for the patient;
a communication network operably coupled with the data center and with a location of a healthcare provider;
the data center operable to provide a plurality of selectable parameters to a healthcare provider through the communication network for scheduling a communication session with a patient;
the data center, utilizing parameters selected by the healthcare provider, contacting a patient through a communication network and conducting a communication session with the patient and providing, to the patient, at least one of educational information, reminders to take a particular action; or a prompt for a response from the patient.

42. The system of claim 41 wherein the patient communication network is one of a telephone network and an Internet network.

43. The system of claim 41 wherein the healthcare provider communication network is an Internet network.

44. The system of claim 41 wherein the educational information includes a tutorial that is played for the patient.

45. The system of claim 41 wherein the reminder to take a particular action includes a reminder about a future appointment with a healthcare provider.

46. The system of claim 41 wherein the reminder to take a particular action includes a reminder about transmitting data from a metering device used by the patient.

47. The system of claim 41 wherein the prompt for a response includes transmitting a series of questions to the patient to be answered.

48. The system of claim 47 wherein the data center further comprises a speech recognition engine for capturing and recording answers to the questions.

49. The system of claim 42 wherein the communication session includes on of an email or instant message.

50. The system of claim 41 wherein the data center conducts a communication session by providing a link to a website to a patient.
